(19) United States
(12) Patent Application Publication
(10) Pub. No.: US 2024/0115107 A1
(43) Pub. Date: Apr. 11, 2024

(54) MOBILE CLEANING DEVICE WITH STERILIZATION FUNCTION

(71) Applicant: KOREA ENVIRONMENTAL INDUSTRY CO., LTD, Daejeon (KR)

(72) Inventor: Sang Yun LEE, Daejeon (KR)

(73) Assignee: KOREA ENVIRONMENTAL INDUSTRY CO., LTD, Daejeon (KR)

(21) Appl. No.: 17/972,075

(22) Filed: Oct. 24, 2022

(30) Foreign Application Priority Data
Oct. 5, 2022 (KR) 10-2022-0127185

(51) Int. Cl. A47L 13/51; A47L 13/502; A47L 13/58; B62B 3/10

(52) U.S. Cl. CPC A47L 13/51; A47L 13/502; A47L 13/58; B62B 3/10; B62B 2202/50

(57) ABSTRACT

Disclosed herein is a mobile cleaning device with a sterilization function. The mobile cleaning device includes: a main body including a body frame having a space part therein, a partition for partitioning the inner space of the body frame into right and left side, and a plurality of wheels mounted below the body frame; a tank unit installed on the partition and filled with washing water; a cleaning unit detachably installed at an upper portion of the body frame and located in the opposite direction to the tank unit with respect to the partition so that an object to be cleaned is positioned thereon; a handle unit to which the washing water filled in the tank unit is selectively supplied so as to perform cleaning operation for the cleaning object positioned in the cleaning unit; a filter unit installed on the partition and located in the same direction as the tank unit with respect to the partition to filter the contaminated washing water used for cleaning the cleaning object in the cleaning unit; and a sterilization unit installed on the partition and located in the same direction as the tank unit with respect to the partition to sterilize the washing water filtered by the filter unit.

### BACKGROUND OF THE INVENTION

### Field of the Invention

The present invention relates to a mobile cleaning device with a sterilization function.

More specifically, the present invention relates to a mobile cleaning device with a sterilization function which can reuse contaminated washing water by purifying washing water used to clean a cleaning object while going through a filter unit and a sterilization unit in order in a case in which the cleaning object is being cleaned in a cleaning unit, thereby preventing an increase of cleaning time due to a waste of a moving line caused by frequent washing of the cleaning object.

### Background Art

A portable cleaning cart (cleaning device) having a separate device capable of holding cleaning articles, such as a rag, or washing or dehydrating the rag is used in a wide cleaning site, such as a hospital, a school or the like, in order to enhance efficiency of cleaning.

Such a cleaning device is mainly movable, and has a space for holding various cleaning articles such as a rag, a garbage bag, and the like, thereby improving cleaning efficiency.

Meanwhile, a mop is a product designed to easily clean the floor of a cleaning site, and has a support plate formed at an end of a rod serving as a handle to support a rag. A rag is fixed to the support plate or is detachably attached to the support plate to perform cleaning.

In this instance, after cleaning, the rag is contaminated and turned into a darkish color or various kinds of dust and wastes are attached to the rag. So, in order to clean again, a user has to wash the rag.

With respect to the above cleaning device, Korean Utility Model Registration No. 20-0284417 discloses a mop cleaning device.

The conventional mop cleaning device washes a mop by using a cleaning plate in a water tub, puts the washed mop in a dehydration tub disposed in the water tub, and dehydrates the mop by pressing the mop while spinning a dehydration plate, thereby performing cleaning and dehydration of the mop in the same place.

The conventional mop cleaning device performs cleaning by supplying hot water or cold water by a water feed tube supplied into the water tub and spraying water through a nozzle. In this instance, the conventional mop cleaning device discharges the used washing water through a drain tube after finishing the cleaning of the mop. So, the conventional mop cleaning device cannot reuse the used washing water after using the washing water once.

### PATENT LITERATURE

### Patent Documents

Korean Utility Model Registration No. 20-0284417 (Jul. 23, 2002)
Korean Patent No. 10-2302456 (Sep. 9, 2021)

### SUMMARY OF THE INVENTION

Accordingly, the present invention has been made to solve the above-mentioned problems occurring in the prior arts, and it is an object of the present invention to provide a mobile cleaning device with a sterilization function which can reuse contaminated washing water by purifying washing water used to clean a cleaning object while going through a filter unit and a sterilization unit in order in a case in which the cleaning object is being cleaned in a cleaning unit, thereby preventing an increase of cleaning time due to a waste of a moving line caused by frequent washing of the cleaning object.

To accomplish the above object, according to the present invention, there is provided a mobile cleaning device with a sterilization function for cleaning and sterilizing an object to be cleaned, including: a main body including a body frame having a space part therein, a partition for partitioning the inner space of the body frame into right and left side, and a plurality of wheels mounted below the body frame; a tank unit installed on the partition and filled with washing water; a cleaning unit detachably installed at an upper portion of the body frame and located in the opposite direction to the tank unit with respect to the partition so that an object to be cleaned is positioned thereon; a handle unit to which the washing water filled in the tank unit is selectively supplied so as to perform cleaning operation for the cleaning object positioned in the cleaning unit; a filter unit installed on the partition and located in the same direction as the tank unit with respect to the partition to filter the contaminated washing water used for cleaning the cleaning object in the cleaning unit; and a sterilization unit installed on the partition and located in the same direction as the tank unit with respect to the partition to sterilize the washing water filtered by the filter unit.

Moreover, the filter unit is installed to be located lower than the cleaning unit and the sterilization unit installed in the main body.

Furthermore, the mobile cleaning device further includes a pump unit configured to flow washing water, wherein the pump unit includes: a first pump for pumping the washing water filtered by the filter unit to the sterilization unit; and a second pump for pumping the washing water filled in the tank unit to the handle unit.

Additionally, the sterilization unit includes: a sterilization tank having a sterilization tank inlet configured to introduce washing water filtered by the filter unit and a sterilization tank outlet configured to discharge the sterilized washing water; and an ultrasonic sterilization module installed on the sterilization tank to perform ultrasonic sterilization operation with respect to the washing water introduced into the sterilization tank through the sterilization tank inlet.

In addition, the main body includes: a through hole penetrating an upper portion of the main body frame to allow a hose of the handle unit to be movable therethrough; and a handle holding member mounted in the through hole of the main body to hold a handle of the handle unit.

Moreover, the cleaning unit includes: a cleaning body having an open upper portion; an inclined surface formed inside the cleaning body to form a washing groove having a shape corresponding to the cleaning object; and a water drain formed at the end of the inclined surface to discharge the contaminated washing water discharged from the handle unit and used for cleaning the object.

Furthermore, the cleaning unit includes: a filter frame mounted below the cleaning body; a first filter detachably mounted at an upper portion of the filter frame in the form of a mesh to filter foreign substances from the washing water discharged through the water drain; and a second filter detachably mounted at a lower portion of the filter frame in the form of a mesh, which has a narrower interval than the mesh of the first filter, to filter the washing water filtered by the first filter.

Additionally, the handle unit includes: a handle body;
a handle extending from an upper portion of the handle body; a protrusion member protruding from a lower portion of the handle body; and an injection hole formed at the lower portion of the handle body to spray washing water supplied from the tank unit.

In addition, the protrusion member includes: a plurality of cleaning protrusions protruding from the rear side of the handle body with respect to the center of the lower portion of the handle body and being spaced apart from one another at predetermined intervals to clean the cleaning object; and a dehydration protrusion formed on the front side with respect to the center of the lower portion of the handle body in a horizontally elongated shape to dehydrate the cleaning object.

The movable cleaning device with the sterilization function according to the present invention can reuse contaminated washing water by purifying washing water used to clean a cleaning object while going through a filter unit and a sterilization unit in order in a case in which the cleaning object is being cleaned in a cleaning unit, thereby preventing an increase of cleaning time due to a waste of a moving line caused by frequent washing of the cleaning object.

Moreover, the movable cleaning device according to the present invention prevent the filtered washing water from flowing backward in the filter unit since the installation position of the filter unit installed inside the main body is lower than those of the tank unit and the cleaning unit.

Furthermore, the movable cleaning device according to the present invention can sufficiently pump the washing water as much as necessary since the washing water filtered by the filter unit is pumped by the first pump to the sterilizing unit, and the washing water filled in the tank unit is pumped to the handle unit by the second pump.

Additionally, the movable cleaning device according to the present invention can remove bacterial-form microorganisms contained in the washing water so as to facilitate reuse of the washing water since operating the ultrasonic sterilization module after the washing water filtered by the filter unit is moved to the sterilization tank.

In addition, the movable cleaning device according to the present invention includes the through hole formed in the main body to penetrate the upper portion of the body frame so that the hose of the handle unit is movable and the handle holding member mounted on the main body to hold a grip of the handle unit, thereby allowing the user to easily use and hold the handle unit.

Moreover, the movable cleaning device according to the present invention includes the inclined surface formed inside the cleaning body to hold the cleaning object and the water drain formed at the end of the inclined surface, thereby easily discharging the contaminated water used for cleaning the object.

Furthermore, according to the present invention, the first filter and the second filter are detachably attached to a filtering frame mounted at the lower portion of the cleaning body. The first filter and the second trainer respectively have mesh net shape having different intervals, thereby performing filtering of the first filter and the second filter in a stepwise manner to facilitate reuse of the washing water.

In addition, according to the present invention, the protrusion member formed on the handle body has the cleaning protrusions for cleaning the cleaning object and the dehydration protrusion for dehydrating the cleaning object, so that the movable cleaning device according to the present invention can clean and dehydrate the cleaning object at the same time according to the use of the handle unit.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIGS. 1A, 1B, and 1C are, respectively, a view illustrating a mobile cleaning device with a sterilization function according to an embodiment of the present invention;

FIG. 2 is a view illustrating a used state of the mobile cleaning device with a sterilization function according to the present invention;

FIG. 3 is a view illustrating a through hole and a handle holding member of the mobile cleaning device according to the present invention;

FIGS. 4A and 4B are, respectively, a view illustrating a filtering frame, a first filter, and a second filter of the mobile cleaning device according to the present invention; and

FIGS. 5A and 5B are, respectively, a view illustrating a handle unit of the mobile cleaning device according to the present invention.

### DETAILED DESCRIPTION OF THE PREFERRED EMBODIMENT

The example embodiments of the present invention are capable of various modifications and alternative forms, and particular embodiments of the present invention will be illustrated in the attached drawings and described in this specification in detail. It should be understood, however, that there is no intent to limit example embodiments of the invention to the particular forms disclosed, but on the contrary, example embodiments of the invention are to cover all modifications, equivalents, and alternatives falling within the technical idea and scope of the present invention.

It will be understood that when an element is referred to as being “connected” or “coupled” to another element, it can be directly connected or coupled to the other element or intervening elements may be present.

In contrast, when an element is referred to as being “directly connected” or “directly coupled” to another element, there are no intervening elements present.

The terms used in the following description are intended to merely describe specific embodiments, but not intended to limit the invention. An expression of the singular number includes an expression of the plural number, so long as it is clearly read differently. The terms such as “include” and “have” are intended to indicate that features, numbers, steps, operations, elements, components, or combinations thereof used in the following description exist and it should thus be understood that the possibility of existence or addition of one or more other different features, numbers, steps, operations, elements, components, or combinations thereof is not excluded.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and/or the present application, and will not be interpreted in an idealized or overly formal sense unless expressly so defined herein.

Hereinafter, the present invention will be described in detail with reference to the accompanying drawings. The terms and words used in the specification and claims must not be limited to typical or dictionary meanings, but must be regarded as concepts selected by the inventor as concepts which best illustrate the present invention, and must be interpreted as having meanings and concepts adapted to the scope and spirit of the present invention to aid in understanding the technology of the present invention. Unless defined otherwise herein, all technical and scientific terms used have the same meaning as commonly understood by one of ordinary skill in the art. In the description, details of well-known features and techniques may be omitted to avoid unnecessarily obscuring the presented embodiments. The attached drawings are provided to sufficiently transfer the spirit of the present invention to those skilled in the art. Additionally, the present invention is not limited to the attached drawings, and may be implemented in various different forms. Moreover, like reference numerals designate like elements throughout the specification. As for reference numerals associated with parts in the drawings, the same reference numerals will refer to the same or like parts throughout the drawings.

The present invention relates to a mobile cleaning device with a sterilization function which can reuse contaminated washing water by purifying washing water used to clean a cleaning object while going through a filter unit and a sterilization unit in order in a case in which the cleaning object is being cleaned in a cleaning unit, thereby preventing an increase of cleaning time due to a waste of a moving line caused by frequent washing of the cleaning object.

Hereinafter, the mobile cleaning device with a sterilization function according to the present invention will be described in detail with reference to the accompanying drawings.

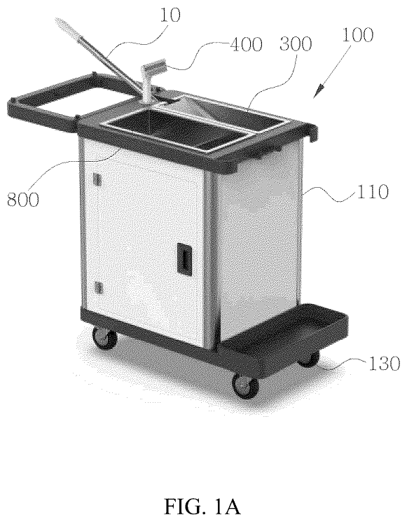

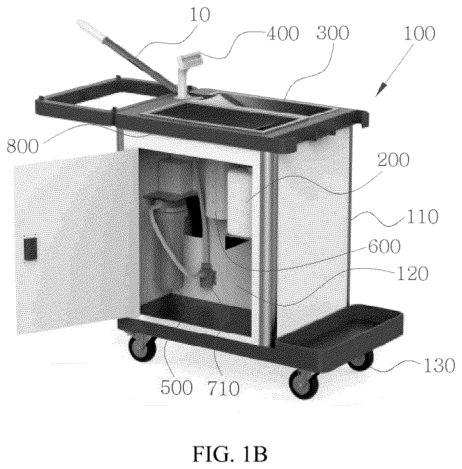

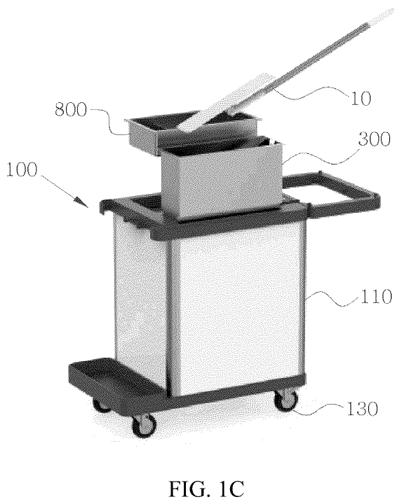

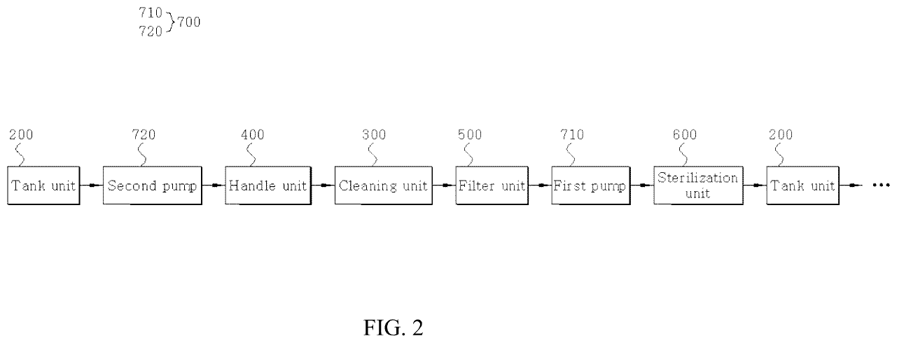

FIGS. 1A, 1B, and 1C are, respectively, a view illustrating a mobile cleaning device with a sterilization function according to an embodiment of the present invention, FIG. 2 is a view illustrating a used state of the mobile cleaning device with a sterilization function according to the present invention.

Referring to FIGS. 1A, 1B and 1C, the mobile cleaning device with a sterilization function according to the present invention includes a main body 100, a tank unit 200, a cleaning unit 300, a handle unit 400, a filter unit 500, and a sterilization unit 600.

In order to clean and sterilize a cleaning object 10, the mobile cleaning device with the sterilization function according to the present invention is configured to directly clean the object on the spot without moving to a restroom or the like. That is, the mobile cleaning device can reuse the used washing water by filtering and sterilizing the contaminated washing water used for cleaning the object 10 on the spot, thereby repeatedly performing cleaning of the cleaning object 10 several times.

The main body 100 includes: a body frame 110 having a space part therein; a partition 120 for partitioning the inner space of the body frame 110 into right and left side; and a plurality of wheels 130 mounted below the body frame 110.

The body frame 110 and the partition 120 form an outward frame of the mobile cleaning device with the sterilization function according to the present invention, and is made of a material having a predetermined strength to prevent a breakage by power or vibration caused by cleaning of the cleaning object 10.

Moreover, since the plurality of wheels 130 mounted below the body frame 110 have a general configuration, a detailed description thereof will be omitted.

The tank unit 200 is installed on the partition 120 and is filled with washing water.

The tank unit 200 serves as a water tank filled with washing water, and is configured to pump the washing water by a pump unit 700 to be described later.

In this instance, the tank unit 200 includes a turbidity sensor (not shown) configured to measure turbidity of the washing water introduced into the tank unit 200 after being used for cleaning the cleaning object 10 in the cleaning unit 300 and being filtered and sterilized by the filter unit 500 and the sterilization unit 600.

The turbidity sensor measures turbidity of the washing water filled in the tank unit 200, and determines whether to reuse the washing water of the tank unit 200 based on the measured turbidity.

In a case in which a numerical value of turbidity measured by the turbidity sensor of the tank unit 200 is more than a predetermined numerical value, the turbidity sensor determines it is impossible to reuse the washing water. In a case in which it is determined that reuse of the washing water is impossible, the turbidity sensor transmits a signal to ring an alarm through a speaker (not shown) or an LED (not shown).

The speaker and the LED have a general configuration, a detailed description thereof will be omitted.

The cleaning unit 300 is detachably installed at an upper portion of the body frame 110 and is located in the opposite direction to the tank unit 200 with respect to the partition 120, and then, the cleaning object 10 is positioned. So, the cleaning unit 300 serves as a space in which the cleaning object 10 is cleaned by the handle unit 400 to be described later.

The cleaning unit 300 includes: a cleaning body 310 having an open upper portion; an inclined surface 320 formed inside the cleaning body 310 to form a washing groove 320a having a shape corresponding to the cleaning object 10; and a water drain 330 formed at the end of the inclined surface 320 to discharge the contaminated washing water discharged from the handle unit 400 and used for cleaning the object 10.

That is, in a state in which the cleaning object 10 is positioned on the inclined surface 320 formed in the cleaning body 310, the cleaning object 10 is easily cleaned by the handle unit 400 to be described later.

Meanwhile, the contaminated washing water discharged through the water drain 330 is filtered through a first filter 350 and the second filter 360, which will be described below, and then, is moved back to the tank unit 200 through the filter unit 500 and the sterilizer 600.

The washing water filled in the tank unit 200 is selectively supplied to the handle unit 400 so that the cleaning object 10 positioned in the cleaning unit 300 is cleaned.

The handle unit 400 is configured to clean the object in a state in which the object 10 is held on the inclined surface 320 of the washing unit 300, so that the user can continuously use the cleaning object 10 in a clean state.

That is, it is preferable that the handle unit 400 has a shape that the user can easily grasp.

The filter unit 500 is installed on the partition 120 and is located in the same direction as the tank unit 200 with respect to the partition 120 to filter the contaminated washing water used for cleaning the cleaning object 10 in the cleaning unit 300.

The filter unit 500 is installed to be located lower than the cleaning unit 300 and the sterilization unit 600 installed in the main body 100.

The filter unit 500 is located lower than the cleaning unit 300 and the sterilization unit 600 to prevent the washing water filtered by the filter unit 500 from flowing backward.

The sterilization unit 600 is installed on the partition 120 and is located in the same direction as the tank unit 200 with respect to the partition 120 to sterilize the washing water filtered by the filter unit 500.

The sterilization unit 600 includes: a sterilization tank 610 having a sterilization tank inlet 611 configured to introduce washing water filtered by the filter unit 500 and a sterilization tank outlet 612 configured to discharge the sterilized washing water; and an ultrasonic sterilization module 620 installed on the sterilization tank 610 to perform ultrasonic sterilization operation with respect to the washing water introduced into the sterilization tank 610 through the sterilization tank inlet 611.

That is, the sterilizer 600 has the ultrasonic sterilization module 620 performing ultrasonic sterilization with respect to the washing water introduced into the sterilization tank 610.

More specifically, when the sterilization tank 610 is filled with washing water of a predetermined capacity, the sterilization unit 600 sterilizes the washing water. The washing water is transferred to the tank unit 200 after the ultrasonic sterilization module 620 performs ultrasonic sterilization as long as a predetermined period of time.

Meanwhile, the mobile cleaning device with the sterilization function according to the present invention further includes a pump unit 700 configured to flow washing water.

The pump unit 700 includes: a first pump 710 for pumping the washing water filtered by the filter unit 500 to the sterilization unit 600; and a second pump 720 for pumping the washing water filled in the tank unit 200 to the handle unit 400.

In this instance, the first pump 710 is configured to promptly pump the washing water filtered by the filter unit 500 to the sterilizing unit 600, and the second pump 720 is configured to pump the washing water according to a control signal transmitted from the handle unit 400.

In addition, the mobile cleaning device with the sterilization function according to the present invention further includes a storage unit 800 which is detachably installed on the upper portion of the body frame 110, is located in the same direction as the tank part 200 with respect to the partition 120, and is adjacent to the washing unit 300.

The storage unit 800 is detachably installed at the upper portion of the body frame 110 like the cleaning unit 300, and stores necessary detergent or mop required for cleaning.

The storage unit 800 is configured to be detachable upwards so that the user can easily inspect the filter unit 500, the sterilization unit 600, and the tank unit 200, which are installed below the storage unit 800.

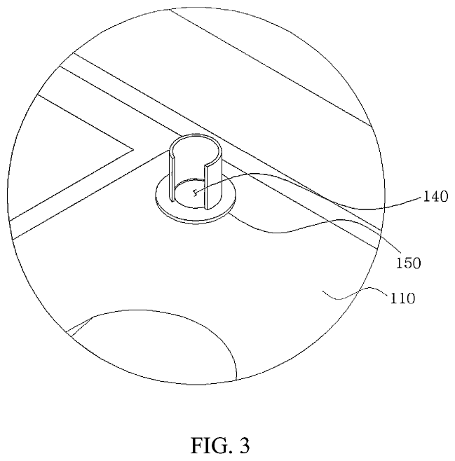

FIG. 3 is a view illustrating a through hole and a handle holding member of the mobile cleaning device according to the present invention.

Referring to FIG. 3, the main body 100 according to the present invention includes: a through hole 140 penetrating an upper portion of the main body frame 110 to allow a hose 450 of the handle unit 400 to be movable therethrough; and a handle holding member 150 mounted in the through hole 140 of the main body 100 to hold a handle 420 of the handle unit 400.

That is, the handle unit 400 is configured to be held on the handle holding member 150. In a case in which the user does not use the handle unit 400, the user holds the handle unit 400 on the handle holding member 150 to safely store the handle unit 400 without shaking even when the mobile cleaning device is moved.

In this instance, the handle holding member 150 has a flow hole in which the hose 450 of the handle unit 400 is movable, so that the handle 420 of the handle unit 400, which will be described later, can be inserted.

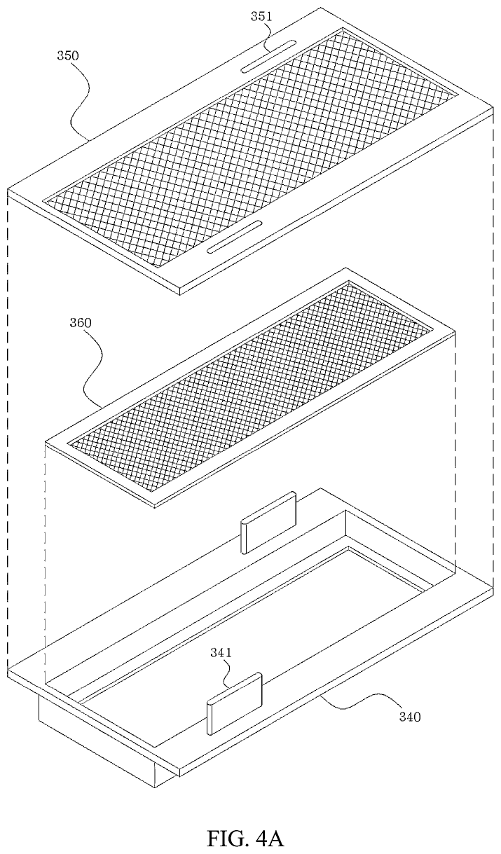

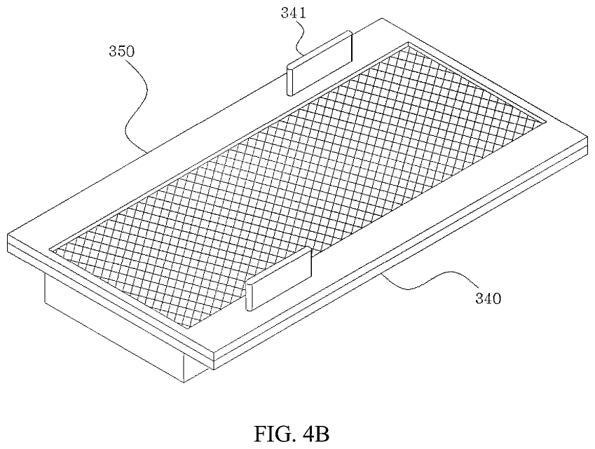

FIGS. 4A and 4B are, respectively, a view illustrating a filtering frame, a first filter, and a second filter of the mobile cleaning device according to the present invention.

Referring to FIGS. 4A and 4B, the cleaning unit 300 according to the present invention includes: a filter frame 340 mounted below the cleaning body 310; a first filter 350 detachably mounted at an upper portion of the filter frame 340 in the form of a mesh to filter foreign substances from the washing water discharged through the water drain 330; and a second filter 360 detachably mounted at a lower portion of the filter frame 340 in the form of a mesh, which has a narrower interval than the mesh of the first filter 350, to filter the washing water filtered by the first filter.

That is, the filter frame 340 is configured such that the first filter 350 and the second filter 360 are detachable and attachable, so that the contaminated washing water discharged through the water drain 330 of the cleaning unit 300 can be filtered by the first filter 350 and the second filter 360.

In this instance, as described above, the second filter 360 is formed in the form of a mesh, which has a narrower interval than the mesh of the first filter 350, so that the washing water is filtered by the second filter 360 after being filtered by the first filter 350. Accordingly, the contaminated washing water discharged through the water drain 330 of the cleaning unit 300 can be filtered in a stepwise manner.

Meanwhile, the filter frame 340 has coupling protrusions 341 protruding upward, and the first filter 350 has coupling grooves 351 formed to correspond to the coupling protrusions, so that the first filter 350 can be detachably mounted to the filter frame 340.

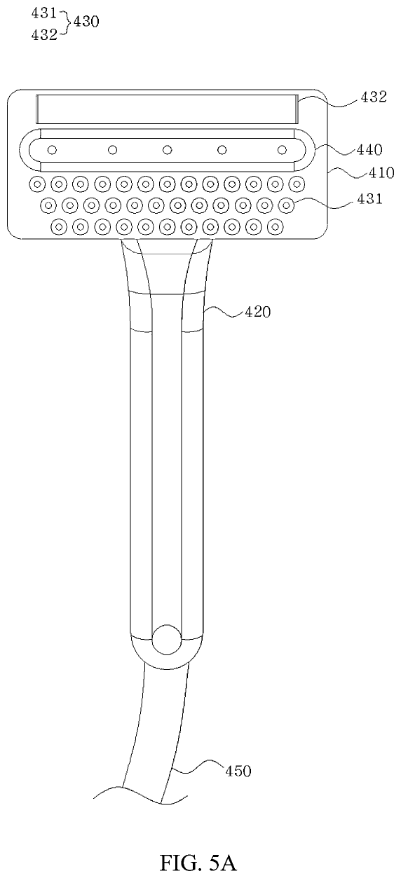

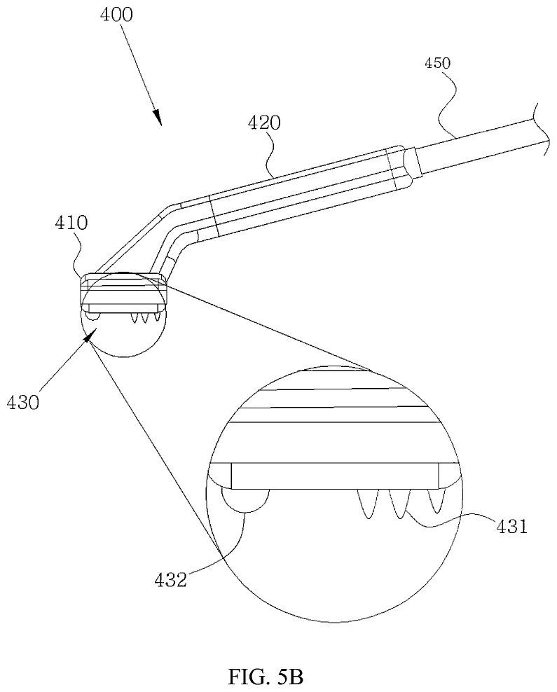

FIGS. 5A and 5B are, respectively, a view illustrating a handle unit of the mobile cleaning device according to the present invention.

Referring to FIGS. 5A and 5B, the handle unit 400 according to the present invention includes: a handle body 410; a handle 420 protruding from an upper portion of the handle body 410 and formed in a cylindrical shape to provide the user with an improved grip feeling so that the user can easily use the handle unit 400; a protrusion member 430 protruding from a lower portion of the handle body 410; and an injection hole 440 formed at the lower portion of the handle body 410 to spray washing water supplied from the tank unit 200.

That is, the user grasps the handle 420, and the mobile cleaning device can easily clean the cleaning object 10 when the protrusion member 430 gets in contact with the cleaning object 10.

More specifically, the mobile cleaning device with the sterilization function according to the present invention sprays washing water through the injection hole 440 in a state in which the user grasps the handle 420, so that the cleaning object 10 gets wet, and is cleaned while the protrusion member 430 gets in contact with the cleaning object 10.

In addition, the protrusion member 430 includes: a plurality of cleaning protrusions 431 protruding from the rear side of the handle body 410 with respect to the center of the lower portion of the handle body 410 and being spaced apart from one another at predetermined intervals to clean the cleaning object 10; and a dehydration protrusion 432 formed on the front side with respect to the center of the lower portion of the handle body 410 in a horizontally elongated shape to dehydrate the cleaning object 10.

That is, the plurality of cleaning protrusions 431 perform cleaning operation for the cleaning object 10 according to forward and backward movement of the handle unit 400 by the user, and the dehydration protrusion 432 performs dehydration operation for the cleaning object 10 according to forward and backward movement of the handle unit 400 by the user.

In other words, the dehydration protrusion 432 is formed at the lower portion of the handle body 410 in the horizontally elongated shape to dehydrate the cleaning object 10 according to forward and backward movement of the handle unit 400.

Although it is illustrated and described that the dehydration protrusion 432 protrudes from the lower portion of the handle body 410 in a semicircular shape not to be rotated, but according to circumstances, the dehydration protrusion 432 may be formed separately and is coupled to the lower portion of the handle body 410 to be rotatable to dehydrate the cleaning object 10 while rotating according to forward and backward movement of the handle unit 400.

The mobile cleaning device with a sterilization function according to the present invention can reuse contaminated washing water by purifying washing water used to clean a cleaning object while going through a filter unit and a sterilization unit in order in a case in which the cleaning object is being cleaned in a cleaning unit, thereby preventing an increase of cleaning time due to a waste of a moving line caused by frequent washing of the cleaning object.

As described above, while the present invention has been particularly shown and described with reference to the various embodiments thereof, it will be understood by those of ordinary skill in the art that the protective scope of the present invention is not limited to the above embodiments and various alternatives, changes, and modifications may be made therein without departing from the technical idea of the present invention.

What is claimed is:

1. A mobile cleaning device with a sterilization function for cleaning and sterilizing an object to be cleaned, comprising:
a main body including a body frame having a space part therein, a partition for partitioning the inner space of the body frame into right and left side, and a plurality of wheels mounted below the body frame;
a tank unit installed on the partition and filled with washing water;
a cleaning unit detachably installed at an upper portion of the body frame and located in the opposite direction to the tank unit with respect to the partition so that an object to be cleaned is positioned thereon;
a handle unit to which the washing water filled in the tank unit is selectively supplied so as to perform cleaning operation for the cleaning object positioned in the cleaning unit;
a filter unit installed on the partition and located in the same direction as the tank unit with respect to the partition to filter the contaminated washing water used for cleaning the cleaning object in the cleaning unit; and
a sterilization unit installed on the partition and located in the same direction as the tank unit with respect to the partition to sterilize the washing water filtered by the filter unit.

2. The mobile cleaning device according to claim 1, wherein the filter unit is installed to be located lower than the cleaning unit and the sterilization unit installed in the main body.

3. The mobile cleaning device according to claim 1, further comprising:
a pump unit configured to flow washing water,
wherein the pump unit includes:
a first pump for pumping the washing water filtered by the filter unit to the sterilization unit; and
a second pump for pumping the washing water filled in the tank unit to the handle unit.

4. The mobile cleaning device according to claim 1, wherein the sterilization unit includes:
a sterilization tank having a sterilization tank inlet configured to introduce washing water filtered by the filter unit and a sterilization tank outlet configured to discharge the sterilized washing water; and
an ultrasonic sterilization module installed on the sterilization tank to perform ultrasonic sterilization operation with respect to the washing water introduced into the sterilization tank through the sterilization tank inlet.

5. The mobile cleaning device according to claim 1, wherein the main body includes:
a through hole penetrating an upper portion of the main body frame to allow a hose of the handle unit to be movable therethrough; and
a handle holding member mounted in the through hole of the main body to hold a handle of the handle unit.

6. The mobile cleaning device according to claim 1, wherein the cleaning unit includes:
a cleaning body having an open upper portion;
an inclined surface formed inside the cleaning body to form a washing groove having a shape corresponding to the cleaning object; and
a water drain formed at the end of the inclined surface to discharge the contaminated washing water discharged from the handle unit and used for cleaning the object.

7. The mobile cleaning device according to claim 6, wherein the cleaning unit includes:
a filter frame mounted below the cleaning body;
a first filter detachably mounted at an upper portion of the filter frame in the form of a mesh to filter foreign substances from the washing water discharged through the water drain; and
a second filter detachably mounted at a lower portion of the filter frame in the form of a mesh, which has a narrower interval than the mesh of the first filter, to filter the washing water filtered by the first filter.

8. The mobile cleaning device according to claim 1, wherein the handle unit includes:
a handle body;
a handle extending from an upper portion of the handle body;
a protrusion member protruding from a lower portion of the handle body; and
an injection hole formed at the lower portion of the handle body to spray washing water supplied from the tank unit.

9. The mobile cleaning device according to claim 8, wherein the protrusion member includes:
a plurality of cleaning protrusions protruding from the rear side of the handle body with respect to the center of the lower portion of the handle body and being spaced apart from one another at predetermined intervals to clean the cleaning object; and
a dehydration protrusion formed on the front side with respect to the center of the lower portion of the handle body in a horizontally elongated shape to dehydrate the cleaning object.
